(19) United States
(12) Patent Application Publication
(10) Pub. No.: US 2022/0104776 A1
(43) Pub. Date: Apr. 7, 2022

(54) APPARATUS AND METHOD FOR ESTIMATING BIO-INFORMATION, AND METHOD OF OPTIMIZING BIO-INFORMATION ESTIMATION MODEL BASED ON TEMPERATURE VARIATION CHARACTERISTIC

(71) Applicant: SAMSUNG ELECTRONICS CO., LTD., Suwon-si (KR)

(72) Inventors: Ka Ram CHOI, Hwaseong-si (KR); So Young Lee, Daejeon (KR); Jun Ho Lee, Incheon (KR); Sang Kyu Kim, Yongin-si (KR)

(73) Assignee: SAMSUNG ELECTRONICS CO., LTD., Suwon-si (KR)

(21) Appl. No.: 17/224,318

(22) Filed: Apr. 7, 2021

(30) Foreign Application Priority Data
Oct. 7, 2020 (KR) 10-2020-0129268

(51) Int. Cl. A61B 5/00; A61B 5/1455; A61B 5/145; A61B 5/01

(52) U.S. Cl. CPC A61B 5/7278; A61B 5/7257; A61B 5/7203; A61B 5/01; A61B 5/14532; A61B 5/4866; A61B 5/14546; A61B 5/1455

(57) ABSTRACT

A method of optimizing a bio-information estimation model by reflecting temperature variation characteristics for each wavelength is disclosed. According to an embodiment of the present disclosure, the method of optimizing a bio-information estimation model includes: obtaining a plurality of spectra according to a temperature variation; obtaining a rate of change in absorbance at each wavelength of the plurality of spectra according to the temperature variation by subtracting a reference spectrum from the plurality of spectra; generating a characteristic matrix for each wavelength based on the rate of change in absorbance at each wavelength according to the temperature variation; obtaining a temperature signal spectrum based on the characteristic matrix for each wavelength; and optimizing a bio-information estimation model based on the temperature signal spectrum.

### CROSS-REFERENCE TO RELATED APPLICATION(S)

This application is based on and claims priority under 35 U.S.C. § 119 to Korean Patent Application No. 10-2020-0129268, filed on Oct. 7, 2020, in the Korean Intellectual Property Office, the disclosure of which is incorporated by reference herein in its entirety.

### BACKGROUND

### 1. Field

The disclosure relates to technology for non-invasively estimating bio-information, and more particularly to technology for estimating bio-information by considering temperature variation characteristics for each wavelength.

### 2 Description of Related Art

Diabetes is a chronic disease that causes various complications and can be difficult to manage, such that people with diabetes are advised to check their blood glucose regularly to prevent complications. In particular, when insulin is administered to control blood glucose levels, the blood glucose levels have to be closely monitored to avoid hypoglycemia and control insulin dosage. An invasive method of finger pricking is generally used to measure blood glucose levels. However, while the invasive method may provide high reliability in measurement, it may cause pain and inconvenience as well as an increased risk of disease and infections due to the use of injection. Recently, research has been conducted on methods of non-invasively estimating bio-information, such as blood glucose, by spectrum analysis using a spectrometer without blood sampling.

### SUMMARY

In one general aspect, there is provided a method of optimizing a bio-information estimation model, the method including: obtaining a plurality of spectra according to a temperature variation; obtaining a rate of change in absorbance at each wavelength of the plurality of spectra according to the temperature variation by subtracting a reference spectrum from the plurality of spectra; generating a characteristic matrix for each wavelength based on the rate of change in absorbance at each wavelength according to the temperature variation; obtaining a temperature signal spectrum based on the characteristic matrix for each wavelength; and optimizing the bio-information estimation model based on the temperature signal spectrum.

In addition, the method of optimizing a bio-information estimation model may further include removing noise from the plurality of spectra based on at least one of differentiation, filtering, asymmetric least square (ALS), detrend, multiplicative scatter correction (MSC), extended multiplicative scatter correction (EMSC), standard normal variate (SNV), mean centering (MC), Fourier transform (FT), orthogonal signal correction (OSC), and Savitzky-Golay (SG) smoothing.

The reference spectrum may include at least one among any one of the plurality of spectra, an average of the plurality of spectra, a spectrum measured in a fasting state, and a spectrum measured in an aqueous solution.

The generating of the characteristic matrix for each wavelength may include converting the rate of change in absorbance at each wavelength according to the temperature variation into vectors, and generating the characteristic matrix for each wavelength according to the temperature variation by sequentially calculating an inner product between an absorbance change vector at a specific wavelength and absorbance change vectors at all wavelengths.

The obtaining of the temperature signal spectrum may include selecting a row or a column from the characteristic matrix for each wavelength according to the temperature variation, and obtaining a spectrum of the row or the column as the temperature signal spectrum.

The obtaining of the temperature signal spectrum may include selecting the row or the column from the characteristic matrix for each wavelength according to the temperature variation, based on a degree of change in spectrum of each row and column of the characteristic matrix.

The obtaining of the temperature signal spectrum may include selecting the row or the column, at which a degree of change in spectrum is greatest, from the characteristic matrix for each wavelength according to the temperature variation.

The optimizing of the bio-information estimation model may include obtaining a plurality of bio-information estimation models optimized according the temperature variation, by updating the bio-information estimation model based on the temperature signal spectrum according to the temperature variation.

Moreover, the method of optimizing a bio-information estimation model may further include generating the bio-information estimation model based on the plurality of spectra.

The bio-information estimation model may be based on classical least square (CLS) or net analyte signal (NAS).

In another general aspect, there is provided an apparatus for estimating bio-information, the apparatus including: a spectrum measurer configured to measure a spectrum from an object of a user; and a processor configured to obtain a temperature signal spectrum, corresponding to a temperature characteristic at a time of measurement of the spectrum, based on characteristic data for each wavelength of the spectrum according to a temperature variation, and to estimate the bio-information based on the measured spectrum by using a bio-information estimation model in which the temperature signal spectrum is reflected.

The spectrum measurer may include: a light source configured to emit light onto the object; and a detector configured to detect light reflected by or scattered from the object.

The processor may obtain the temperature signal spectrum, corresponding to the temperature characteristic at the time of measurement of the spectrum, based on at least one of a similarity and a variance between a plurality of temperature signal spectra, obtained based on the characteristic data for each wavelength, and the measured spectrum, or by using a result of a statistical test.

The similarity may include at least one of Euclidean distance, Pearson correlation coefficient, Spearman correlation coefficient, and Cosine similarity.

The statistical test may include at least one of t-test, z-test, and ANOVA test.

The processor may detect a change in the temperature characteristic at the time of measurement of the spectrum, and based on detecting the change in the temperature characteristic, the processor may obtain the temperature signal spectrum corresponding to the temperature characteristic at the time of measurement of the spectrum.

The processor may detect the change in the temperature characteristic, based on at least one of whether there is a non-temperature dependent wavelength in a specific wavelength range, whether a non-temperature dependent wavelength is shifted according to an increase in the temperature variation, and whether a range of wavelengths shorter than a first wavelength increases and a range of wavelengths longer than a second wavelength decreases.

The processor may obtain the bio-information estimation model, in which the temperature signal spectrum is reflected, by updating a reference bio-information estimation model based on the temperature signal spectrum corresponding to the temperature characteristic at the time of measurement of the spectrum.

The processor may obtain a plurality of spectra as training data from the user, and may generate the characteristic data for each wavelength based on the training data.

The processor may obtain a rate of change in absorbance at each wavelength by subtracting a reference spectrum from each of the plurality of spectra, and may generate the characteristic data for each wavelength based on the obtained rate of change in absorbance at each wavelength.

The processor may estimate a relative temperature change trend at the time of measurement of the spectrum compared to a reference time, based on the temperature signal spectrum corresponding to the temperature characteristic at the time of measurement of the spectrum.

The bio-information may include one or more of an antioxidant-related substance, blood glucose, triglyceride, cholesterol, calories, protein, carotenoid, lactate, and uric acid.

In another general aspect, there is provided a method of estimating bio-information, the method including: measuring a spectrum from an object of a user; obtaining a temperature signal spectrum, corresponding to a temperature characteristic at a time of measurement of the spectrum, based on characteristic data for each wavelength of the spectrum according to a temperature variation; and estimating the bio-information based on the spectrum by using a bio-information estimation model in which the obtained temperature signal spectrum is reflected.

The obtaining of the temperature signal spectrum may include obtaining the temperature signal spectrum, corresponding to the temperature characteristic at the time of measurement of the spectrum, based on at least one of a similarity and a variance between a plurality of temperature signal spectra, obtained based on the characteristic data for each wavelength according to the temperature variation, and the measured spectrum, or by using a result of a statistical test.

The obtaining of the temperature signal spectrum may include detecting a change in the temperature characteristic at the time of measurement of the spectrum, and based on detecting the change in the temperature characteristic, obtaining the temperature signal spectrum.

The detecting of the change in the temperature characteristic may include detecting the change in the temperature characteristic, based on at least one of whether there is a non-temperature dependent wavelength in a specific wavelength range, whether a non-temperature dependent wavelength is shifted according to an increase in temperature variation, and whether a range of wavelengths shorter than a first wavelength increases and a range of wavelengths longer than a second wavelength decreases.

In addition, the method of estimating bio-information may further include obtaining the bio-information estimation model, in which the temperature signal spectrum is reflected, by updating a reference bio-information estimation model based on the temperature signal spectrum corresponding to the temperature characteristic at the time of measurement of the spectrum.

Moreover, the method of estimating bio-information may further include: obtaining a plurality of spectra, measured from the user, as training data; and generating the characteristic data for each wavelength based on the obtained training data.

The generating of the characteristic data for each wavelength may include: obtaining a rate of change in absorbance at each wavelength by subtracting a reference spectrum from each of the plurality of spectra; and generating the characteristic data for each wavelength based on the obtained rate of change in absorbance at each wavelength.

Furthermore, the method of estimating bio-information may further include estimating a relative temperature change trend at the time of measurement of the spectrum compared to a reference temperature, based on the temperature signal spectrum corresponding to the temperature characteristic at the time of measurement of the spectrum.

Additional aspects will be set forth in part in the description which follows and, in part, will be apparent from the description, or may be learned by practice of the presented embodiments.

### BRIEF DESCRIPTION OF THE DRAWINGS

The above and other aspects and features of certain embodiments of the present disclosure will be more apparent from the following description taken in conjunction with the accompanying drawings, in which:

FIG. 1 is a block diagram illustrating an apparatus for estimating bio-information according to an example embodiment of the present disclosure;

FIG. 2 is a block diagram illustrating a configuration of a processor according to an example embodiment of the present disclosure;

FIGS. 3A and 3B are diagrams illustrating a change in spectrum according to a temperature variation;

FIGS. 4A to 4G are diagrams explaining examples of optimizing a bio-information estimation model;

FIGS. 5A to 5C are diagrams explaining a change in temperature characteristics;

FIG. 6 is a block diagram illustrating an apparatus for estimating bio-information according to another example embodiment of the present disclosure;

FIG. 7 is a flowchart illustrating a method of estimating bio-information according to an example embodiment of the present disclosure;

FIG. 8 is a flowchart illustrating an example of optimizing a bio-information estimation model;

FIGS. 9 to 11 are diagrams illustrating examples of estimating bio-information; and

FIG. 12 is a wearable device according to an example embodiment of the present disclosure.

Throughout the drawings and the detailed description, unless otherwise described, the same drawing reference numerals will be understood to refer to the same elements, features, and structures. The relative size and depiction of these elements, features, and structures may be exaggerated for clarity, illustration, and convenience.

### DETAILED DESCRIPTION

Details of example embodiments are included in the following detailed description and drawings. Features of the present disclosure, and a method of achieving the same will be more clearly understood from the following embodiments described in detail with reference to the accompanying drawings. Throughout the drawings and the detailed description, unless otherwise described, the same drawing reference numerals will be understood to refer to the same elements, features, and structures.

It will be understood that, although the terms “first,” “second,” etc. may be used herein to describe various elements, these elements should not be limited by these terms. These terms are only used to distinguish one element from another. Any references to the singular form of a term may include the plural form of the term unless expressly stated otherwise. In addition, unless explicitly described to the contrary, an expression such as “comprising” or “including” will be understood to imply the inclusion of the stated elements but not the exclusion of any other elements. Also, the terms, such as “unit,” “module,” etc., should be understood as a unit that performs at least one function or operation and that may be embodied as hardware, software, or a combination thereof.

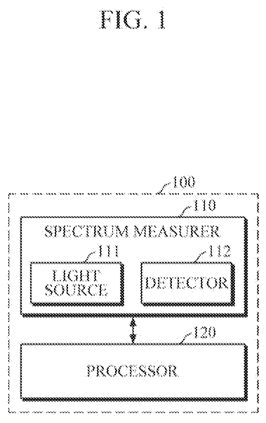

FIG. 1 is a block diagram illustrating an apparatus for estimating bio-information according to an embodiment of the present disclosure.

Referring to FIG. 1, the apparatus 100 for estimating bio-information includes a spectrum measurer 110 and a processor 120.

The spectrum measurer 110 may measure a spectrum from an object in an in-vivo environment or in an in-vitro environment. The spectrum measurer 110 may include, for example, a spectrometer for measuring spectra over a wide wavelength range. In this case, the spectrometer may be used with various spectroscopic techniques, such as Infrared spectroscopy using near-infrared light or mid-infrared light, Raman spectroscopy, and the like. In another example, the spectrum measurer 110 may include an optical sensor for measuring spectra over a narrow wavelength range.

The spectrometer or the optical sensor may include one or more light sources 111 for emitting light onto an object, and one or more detectors 112 for detecting light scattered or reflected from the object. The light source 111 may include a light emitting diode (LED), a laser diode (LD), a phosphor, and the like. The detector 112 may include a photo diode, a photo transistor (PTr), an image sensor (e.g., complementary metal-oxide-semiconductor (CMOS) image sensor), and the like, but is not limited thereto.

The spectrum measurer 110 may measure a spectrum (hereinafter referred to as a “first spectrum”) for calibrating a bio-information estimation model. The spectrum measurer 110 may measure the first spectrum according to a temperature variation at predetermined time intervals by changing temperature of an aqueous solution or temperature of a composite material similar to skin components. Alternatively, the spectrum measurer 110 may measure a plurality of first spectra from a user's skin in a changing temperature environment. For example, by gradually increasing temperature from a reference temperature, the spectrum measurer 110 may measure the first spectra every time temperature is changed by a predetermined value. For example, the spectrum measurer 110 may measure the first spectra every time a temperature variation ΔT increases by 0.1° C. from temperature at a current measurement time. However, the first spectra are not limited thereto.

In addition, the spectrum measurer 110 may measure a spectrum (hereinafter referred to as a “second spectrum”) for estimating bio-information from a user's skin.

The processor 120 may be electrically connected to the spectrum measurer 110 to control the spectrum measurer 110. The processor 120 may receive the first spectrum from the spectrum measurer 110, and may calibrate a bio-information estimation model based on the received first spectrum. Further, the processor 120 may receive the second spectrum and may estimate bio-information based on the received second spectrum. In this case, the bio-information may include antioxidant-related substances, blood glucose, triglyceride, cholesterol, calories, protein, carotenoid, lactate, uric acid, and the like, but the bio-information is not limited thereto. For convenience of explanation, the following description will be given using blood glucose as an example.

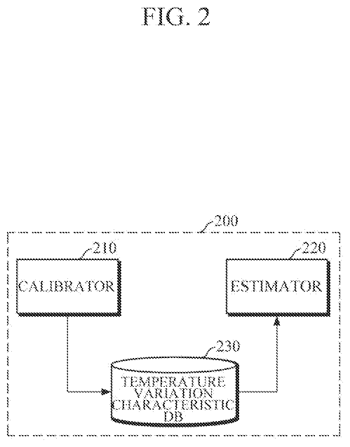

FIG. 2 is a block diagram illustrating a configuration of a processor according to an embodiment of the present disclosure.

Referring to FIG. 2, a processor 200 according to an embodiment may include a calibrator 210 and an estimator 220.

The calibrator 210 may control the spectrum measurer 110 to calibrate a bio-information estimation model. Based on receiving the first spectrum from the spectrum measurer 110, the calibrator 210 may calibrate a bio-information estimation model by using the received first spectrum as training data.

Based on receiving the first spectrum from the spectrum measurer 110, the calibrator 210 may remove noise from the first spectrum based on, as examples, one or more of the following: differentiation, filtering, asymmetric least square (ALS), detrend, multiplicative scatter correction (MSC), extended multiplicative scatter correction (EMSC), standard normal variate (SNV), mean centering (MC), Fourier transform (FT), orthogonal signal correction (OSC), and Savitzky-Golay (SG) smoothing.

The calibrator 210 may generate a bio-information estimation model personalized to a user based on the first spectrum, obtained from the user's skin at a reference time, or the first spectrum obtained using a simulated solution of skin components. In this case, the reference time may be a fasting time, but is not limited thereto.

For example, the calibrator 210 may generate a blood glucose estimation model based on the first spectrum by linear regression, such as classical least square (CLS), net analyte signal (NAS), and the like, or by machine learning. The calibrator 210 may extract a background signal from the first spectrum by using principal component analysis (PCA), independent component analysis (ICA), non-negative matrix factorization, auto-encoding, and the like, and may generate a blood glucose estimation model based on the Lambert-Beer law by using the extracted background signal, a preset blood glucose absorption coefficient, and a light travel path.

The calibrator 210 may optimize a bio-information estimation model by using a plurality of first spectra continuously measured from an aqueous solution or a user's skin in a changing temperature environment, or a plurality of first spectra measured from a user's skin under the condition of various temperature characteristics (e.g., cold weather, hot weather, room temperature, outdoor, indoor, etc.).

For example, the calibrator 210 may obtain a temperature signal spectrum (hereinafter referred to as a “first temperature signal spectrum”) according to a temperature variation by using the plurality of first spectra measured according to a temperature variation. Further, the calibrator 210 may obtain a bio-information estimation model optimized for each temperature variation characteristic, by updating a reference bio-information estimation model based on the obtained first temperature signal spectrum. In this case, the reference bio-information estimation model may be an estimation model before temperature variation characteristics are reflected therein, and may be an estimation model which may be universally applied or may be an estimation model personalized to a user as described above.

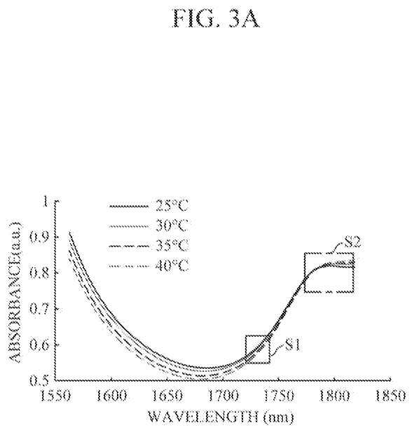

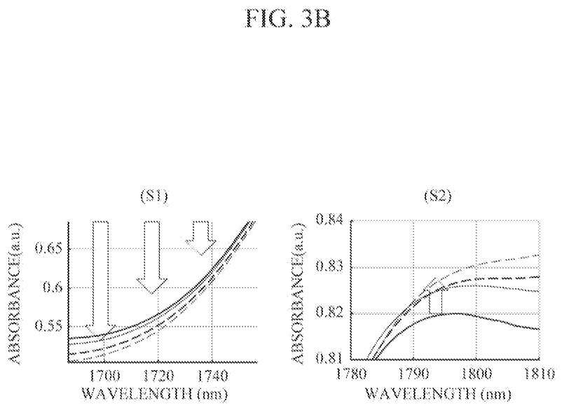

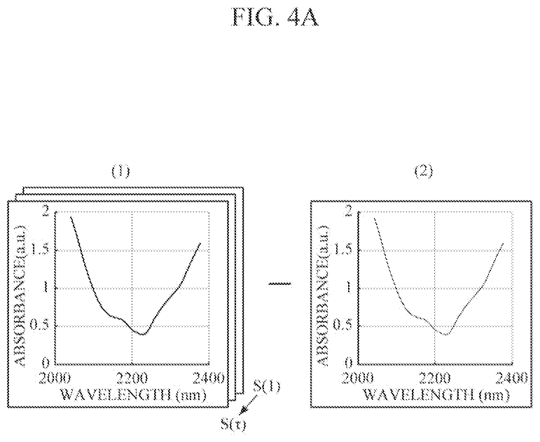

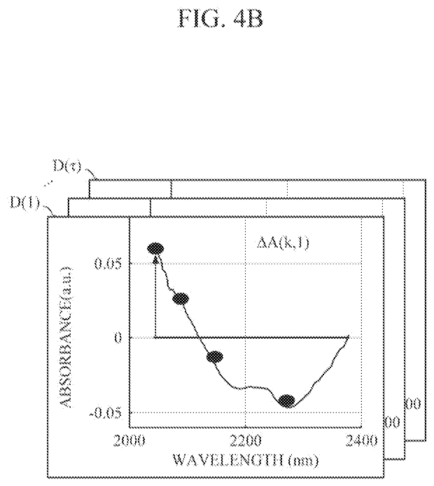

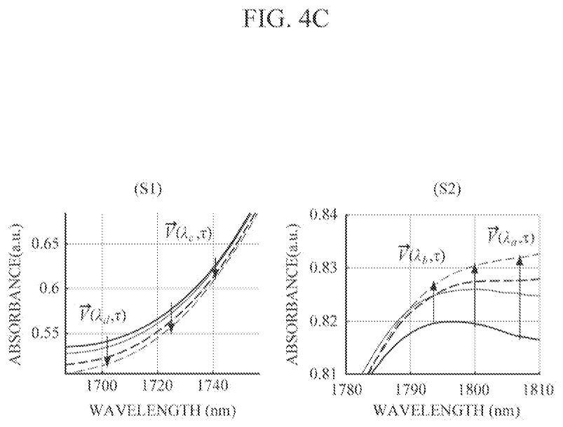

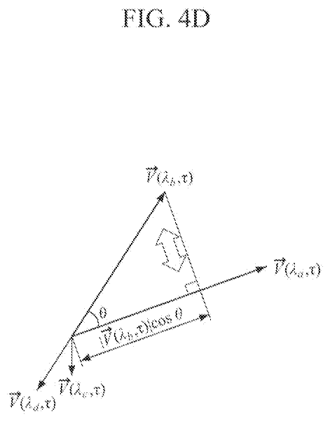

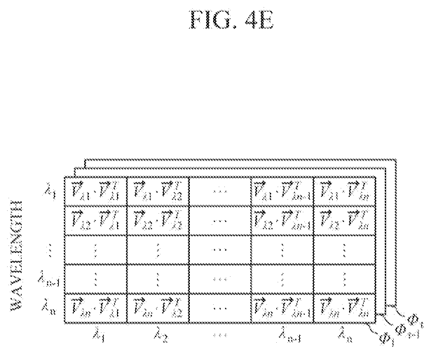

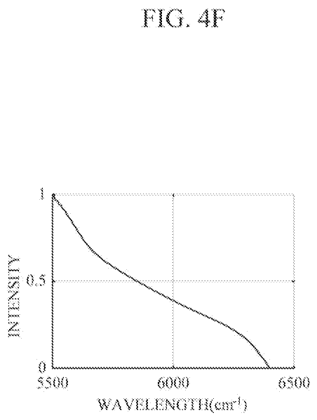

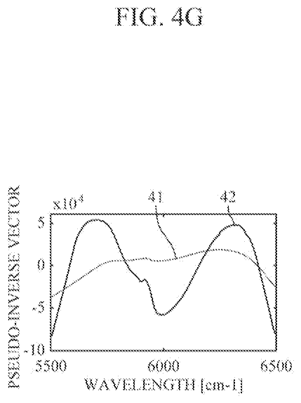

Hereinafter, an operation of optimizing a bio-information estimation model will be described with reference to FIGS. 3A to 4F.

FIGS. 3A and 3B are diagrams illustrating a change in spectrum according to a temperature variation.

FIG. 3A illustrates spectra obtained by changing temperature of an aqueous solution. FIG. 3B is an enlarged view of a first wavelength range S1 and a second wavelength range S2 in the first spectrum of FIG. 3A. Referring to FIG. 3B, it can be seen that as temperature gradually increases in the first wavelength range S1, absorbance gradually decreases; and as temperature gradually increases in the second wavelength range S2, absorbance increases. Accordingly, a change in spectrum at each wavelength varies according to a temperature variation, such that by reflecting wavelength characteristics according to a temperature variation at the time of measurement of bio-information, accuracy in estimating bio-information may be improved.

FIGS. 4A to 4F are diagrams explaining an example of generating characteristic data for each wavelength according to a temperature variation, and obtaining a temperature signal spectrum.

FIG. 4A illustrates first spectra 1 measured at each time when temperature changes, and a reference spectrum 2. Referring to FIG. 4A, the calibrator 210 may calculate a rate of change in absorbance at each wavelength according to a temperature variation by subtracting the reference spectrum 2 from each of the measured first spectra 1. In this case, the reference spectrum 2 may be a spectrum measured at a calibration time, such as a spectrum measured while a user is in a fasting state, a spectrum measured using an aqueous solution, or a spectrum obtained based on the plurality of first spectrum 1. For example, the reference spectrum 2 may be a spectrum measured at any one time (e.g., first time) among the plurality of first spectra 1, an average of all the measured first spectra, or an average of spectra in a specific range, but is not limited thereto.

FIG. 4B illustrates differential spectra obtained by subtracting the reference spectrum 2 from the first spectra 1. As illustrated in FIG. 4B, each of the differential spectra D(1), . . . , and D(τ) may include information on a rate of change in absorbance at each wavelength for the respective first spectra S(1), . . . , and S(τ). For example, ΔA(k, 1) denotes a rate of change in absorbance at each wavelength for the first spectrum measured at a first time, when compared to the reference spectrum. In this case, k denotes a wavelength index, and 1 denotes a spectrum measured at a first time. As illustrated in FIG. 4C, it can be seen from the rate of change in absorbance at each wavelength that non-linear characteristics are shown for each wavelength according to a temperature variation.

As illustrated in FIGS. 4C and 4D, specific wavelengths λₐ, λ_{b}, λ_{c}, and λ_{d} in the first wavelength range S1 and the second wavelength range S2 of the first spectra may be expressed in vectors such as, for example, \vec{V}(λₐ, τ), \vec{V}(λ_{b}, τ), \vec{V}(λ_{c}, τ) and, \vec{V}(λ_{d}, τ), having different rates of change in absorbance and different directions of change according to a temperature variation. As described above, by converting rates of change in absorbance at each wavelength according to a temperature variation into vectors, and by using the absorbance change vectors at each wavelength, the calibrator 210 may generate characteristic data for each wavelength according to each of temperature variation characteristics.

FIG. 4E illustrates characteristic matrices φ₁, . . . , φ_{τ-1}, and φ_{τ} as an example of characteristic data for each wavelength according to a temperature variation. As represented by the following Equation 1, by sequentially calculating an inner product between an absorbance change vector at a specific wavelength and absorbance change vectors at all the wavelengths in the first spectrum measured at a specific time, the calibrator 210 may generate the characteristic matrices φ₁, . . . , φ_{τ-1}, and φ_{τ} for each wavelength according to the temperature variation characteristics. Further, the generated characteristic matrices for each wavelength may be stored in a temperature variation characteristic DB 230.

Φ(τ)=\vec{V}(λᵢ,τ)·\vec{V}(λₖ,τ)^{T} (1≤i≤n,1≤k≤n) [Equation 1]

Herein, Φ(τ) denotes a characteristic matrix for each wavelength in the first spectrum measured at a specific time T; n denotes the number of wavelengths; \vec{V}(λᵢ, τ) denotes the absorbance change vector at a wavelength k; in the first spectrum measured at the specific time τ; and \vec{V}(λₖ, τ)^{T} denotes a transposed vector of an absorbance change vector at a wavelength λₖ in the first spectrum measured at the specific time T.

That is, as illustrated in FIG. 4E, by using, as a reference wavelength, the first wavelength λ₁ in the first spectrum measured at the first time, the calibrator 210 may sequentially calculate an inner product between an absorbance change vector \vec{V}_{λ1} at the reference wavelength and the absorbance change vectors \vec{V}_{λ1}^{T}, . . . , and \vec{V}_{λn}^{T} at all wavelengths, and may arrange the resulting vectors in a first row. As described above, by sequentially changing the reference wavelength until the last wavelength λₙ, the calibrator 210 may calculate an inner product between the absorbance change vector at the reference wavelength and the absorbance change vectors at all wavelengths, and may arrange the resulting vectors in each row.

As described above, based on obtaining characteristic matrices for each wavelength, the calibrator 210 may obtain a first temperature signal spectrum according to a temperature variation, based on the characteristic matrices for each wavelength. For example, the calibrator 210 may select a row/column, at which a degree of change in spectrum is greatest, from the characteristic matrices φ₁, . . . , φ_{τ-1}, and φ_{τ} for each wavelength according to the respective temperature variation characteristics, and may obtain values in the selected row/column as the first temperature signal spectra. FIG. 4F illustrates one of the obtained first temperature signal spectra.

The calibrator 210 may optimize a bio-information estimation model by reflecting the obtained first temperature signal spectra for each temperature variation characteristic in a reference bio-information estimation model. The following Equation 2 represents an example of a blood glucose estimation model.

S=ε_{g}·Lt·ΔC+Σ_{ν=1}^{k}(bᵥBᵥ)+bₜₑₘₚSₜₑₘₚ

K=[ε_{g},Σ_{ν=1}^{k}Bᵥ,Sₜₑₘₚ]

ΔC=[(K^{T}×K)⁻¹×K^{T}]×S Equation 2

Herein, S denotes a spectrum measured for estimating blood glucose; ε_{g}·Lt·ΔC denotes a blood glucose signal; ε_{g} denotes a preset blood glucose absorption coefficient; Lt denotes a unit light path; ΔC denotes a variation in blood glucose to be obtained; Bᵥ denotes the background signal, in which k denotes the number of background signals, such as skin component signals, and bᵥ denotes a coefficient of the respective background signals; Sₜₑₘₚ denotes the first temperature signal spectrum obtained as described above; bₜₑₘₚ denotes a coefficient of the first temperature signal spectrum; K denotes the blood glucose estimation model; and [K^{T}×K)⁻¹×K^{T}] denotes a pseudo-inverse vector.

By reflecting the first temperature signal spectra for each temperature variation characteristic, the calibrator 210 may obtain a bio-information estimation model optimized for each temperature variation characteristic. FIG. 4G illustrates a blood glucose estimation model 41 before consideration of the temperature variation characteristics, and a blood glucose estimation model 42 after consideration of the temperature variation characteristics. As illustrated in FIG. 4G, by considering characteristics for each wavelength according to a temperature variation at the time of estimation of blood glucose, accuracy in estimating blood glucose may be improved.

Examples of operations of the calibrator 210 are described above, which includes generating characteristic data for each wavelength, generating the first temperature signal spectra according to a temperature variation, and optimizing a bio-information estimation model by using the first temperature signal spectra. However, the operations of the calibrator 210 are not limited thereto, and the calibrator 210 may perform only the operation of generating characteristic data for each wavelength, or may perform only the operations of generating characteristic data for each wavelength and generating the first temperature signal spectra.

Referring back to FIG. 2, the estimator 220 may control the spectrum measurer 110 in response to a request for estimating bio-information. Based on receiving the second spectrum for estimating bio-information from the spectrum measurer 110, the estimator 220 may estimate bio-information by using the received second spectrum and the bio-information estimation model optimized by the calibrator 210.

For example, the estimator 220 may obtain, from the temperature variation characteristic DB 230, a temperature signal spectrum (hereinafter referred to as a “second temperature signal spectrum”), corresponding to a temperature variation characteristic at the time of measurement of the second spectrum, among the plurality of first temperature signal spectra generated by the calibrator 210. Alternatively, if the calibrator 210 performs only the operation of generating characteristic data for each wavelength, the estimator 220 may obtain a plurality of first temperature signal spectra based on the characteristic data for each wavelength which are stored in the temperature variation characteristic DB 230, and may obtain the second temperature signal spectrum among the obtained first temperature signal spectra.

For example, the estimator 220 may determine a first temperature signal spectrum, having a similarity to the second spectrum being greater than or equal to a predetermined threshold value (e.g., 0.9), as the second temperature signal spectrum among the first temperature signal spectra stored in the temperature variation characteristic DB 230. In this case, the similarity may include Euclidean distance, Pearson correlation coefficient, Spearman correlation coefficient, Cosine similarity, and the like, but is not limited thereto.

In another example, the estimator 220 may calculate a variance between the first temperature signal spectra, stored in the temperature variation characteristic DB 230, and the second spectrum, and may obtain a first temperature signal spectrum, having a variance being greater than or equal to a predetermined value at a predetermined wavelength, such as at the shortest wavelength, as the second temperature signal spectrum.

In yet another example, by using statistical test methods, such as t-test, z-test, and ANOVA test, the estimator 220 may obtain the second temperature signal spectrum among the first temperature signal spectra.

Based on obtaining the second temperature signal spectrum, the estimator 220 may extract an optimized bio-information estimation model from the temperature variation characteristic DB 230 based on the second temperature signal spectrum. The estimator 220 may estimate bio-information based on the extracted bio-information estimation model and the second spectrum. If the calibrator 210 does not perform the operation of optimizing a bio-information estimation model, the estimator 220 may correct the second spectrum based on the second temperature signal spectrum, and may estimate bio-information by using the corrected second spectrum and the reference bio-information estimation model.

Based on obtaining the second spectrum from the object, the estimator 220 may detect a change in temperature characteristics at the time of measurement of the second spectrum. The estimator 220 may determine whether a temperature variation at the time of measurement of the second spectrum is greater than or equal to a predetermined threshold value compared to a reference temperature. If the temperature variation is greater than or equal to the predetermined threshold value, the estimator 220 may perform the aforementioned operation of obtaining the estimated temperature signal spectrum; and if the temperature variation is less than the predetermined threshold value, the estimator 220 may estimate bio-information by using the reference bio-information estimation model.

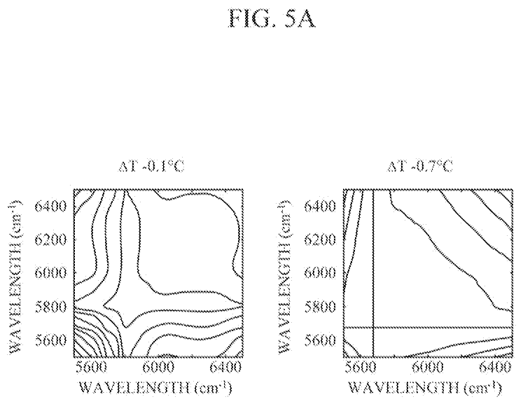

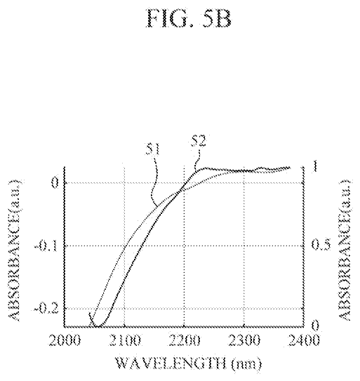

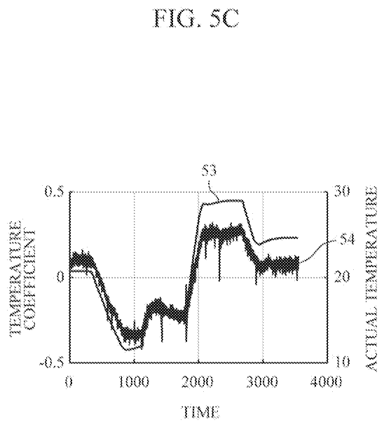

FIG. 5A is a diagram illustrating an example of converting characteristic matrices for each wavelength according to a temperature variation into a two-dimensional (2D) map. For example, a left view shows a 2D map of characteristic matrices for each wavelength when a temperature variation ΔT between temperature at the time of measurement (e.g., 37.5° C.) and a reference temperature (e.g., 37.6° C.) is −0.1° C.; and a right view shows a 2D map of characteristic matrices for each wavelength when a temperature variation ΔT between temperature at the time of measurement (e.g., 36.8° C.) and the reference temperature (e.g., 37.6° C.) is −0.7° C. As illustrated in FIG. 5A, by analyzing the characteristic matrices for each wavelength according to a temperature variation, it is possible to detect a change in temperature characteristics at the time of measurement of the second spectrum compared to the reference temperature.

For example, based on obtaining the second spectrum, the estimator 220 may generate characteristic matrices for each wavelength in the second spectrum by using the second spectrum and a reference spectrum, as described above. In this case, the reference spectrum may be a spectrum measured from a user at the time of calibration. Alternatively, based on a bio-information estimation history of a user, the estimator 220 may obtain the reference spectrum from among second spectra measured at previous times of measurement of bio-information. For example, the estimator 220 may obtain a first spectrum, an intermediate spectrum, or a last spectrum as the reference spectrum from among second spectra obtained within a predetermined period of time from a current time, or may obtain an average of second spectra, obtained within a predetermined period of time, as the reference spectrum. However, the reference spectrum is not limited thereto.

Based on obtaining the characteristic matrices for each wavelength in the second spectrum, the estimator 220 may analyze the characteristic matrices for each wavelength in the second spectrum; and if the characteristic matrices satisfy predetermined criteria, the estimator 220 may detect a change in a temperature characteristic at the time of measurement of the second spectrum.

For example, the estimator 220 may determine that the temperature characteristic is changed based on whether there is a non-temperature dependent wavelength in a specific wavelength range, such as if there is a point at which a wavelength change rate is “0” in a wavelength range of 2100 nm to 2200 nm; or whether a non-temperature dependent wavelength compared to a reference temperature is shifted to a long wavelength; or whether a range of wavelengths shorter than a first wavelength (e.g., 2100 nm) increases and a range of wavelengths longer than a second wavelength (e.g., 2300 nm) decreases. In this case, the specific wavelength range, the first wavelength, and the second wavelength are not limited to the illustrated embodiment, and may be set appropriately according to temperature criteria and the like.

In another example, in a variance spectrum of the characteristic matrix for each wavelength of the second spectrum, if a variance of a specific wavelength (e.g., shortest wavelength) is greater than or equal to a predetermined threshold value, the estimator 220 may determine that a temperature characteristic is changed. In this case, referring to FIG. 4E, the variance spectrum may be a spectrum of diagonal elements \vec{V}_{λ1}·\vec{V}_{λ1}^{T}, . . . , and \vec{V}_{λn}·\vec{V}_{λn}^{T} of the characteristic matrix for each wavelength, but is not limited thereto.

The estimator 220 may estimate a temperature change trend at the time of estimation of bio-information compared to a reference time, by using the temperature variation characteristic DB 230. The estimator 220 may estimate the temperature change trend along with, or separately from, the estimation of bio-information.

For example, FIG. 5B illustrates a first temperature signal spectrum 51 of the temperature variation characteristic DB 230, and a spectrum 52 extracted from the second spectrum, in which absorbance of the first spectrum is shown on the right side of the Y axis, and absorbance of the second spectrum is shown on the left side of the Y axis. Referring to FIG. 5B, in the case where the spectrum 52, having a correlation with the first temperature signal spectrum 51 being greater than a predetermined threshold value, of the temperature variation characteristic DB 230 is extracted from the second spectrum, the estimator 220 may estimate a relative temperature change trend at the time of bio-information estimation based on the temperature variation characteristic of the first temperature signal spectrum 51. In other words, if the first temperature signal spectrum 51 has a temperature variation characteristic indicative of a temperature increase by 0.5° C. from a reference temperature, such as if the first temperature signal spectrum 51 is obtained from the first spectrum measured at a time when temperature increases by 0.5° C. from the reference temperature, the estimator 220 may estimate that a relative temperature change trend at the time of bio-information estimation shows a temperature increase by 0.5° C. from the reference temperature. FIG. 5C illustrates a correlation between estimated values of a relative temperature change trend 54 and actual temperature values 53, measured at a plurality of times. It can be seen from FIG. 5C that the correlation between the estimated values and the actual temperature values is high.

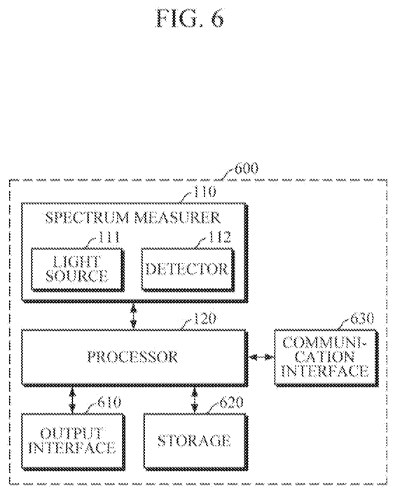

FIG. 6 is a block diagram illustrating an apparatus for estimating bio-information according to another embodiment of the present disclosure.

Referring to FIG. 6, the apparatus 600 for estimating bio-information includes the spectrum measurer 110, the processor 120, an output interface 610, a storage 620, and a communication interface 630. The spectrum measurer 110 may include one or more light sources 111 and one or more detectors 112. The spectrum measurer 110 and the processor 120 are described above, such that description thereof will be omitted.

The output interface 610 may provide processing results of the processor 120 for a user. For example, the output interface 610 may display an estimated bio-information value on a display. In this case, if the estimated bio-information value falls outside of a normal range, the output interface 610 may provide a user with warning information by changing color, line thickness, etc., or displaying the abnormal value along with a normal range, so that the user may easily recognize the abnormal value. Further, along with or without the visual display, the output interface 610 may provide the estimated bio-information value in a non-visual manner by voice, vibrations, tactile sensation, and the like, using a voice output module such as a speaker, or a haptic module, and the like.

The storage 620 may store reference information for estimating bio-information. In this case, the reference information may include user characteristic information, such as a user's age, gender, health condition, and the like. Further, the reference information may include a bio-information estimation model, a reference spectrum, a reference temperature, and the like. In addition, the storage 620 may store processing results of the spectrum measurer 110 and/or the processor 120. For example, the storage 620 may store spectra measured by the spectrum measurer 110, characteristic data for each wavelength which are generated by the processor 120, the first temperature signal spectrum and the second temperature signal spectrum, which are measured according to a temperature variation, and the like. However, the information is not limited thereto.

The storage 620 may include at least one storage medium of a flash memory type memory, a hard disk type memory, a multimedia card micro type memory, a card type memory (e.g., a secure digital (SD) memory, an extreme digital (XD) memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

The communication interface 630 may communicate with an external device to transmit and receive various data, such as spectra, characteristic data for each wavelength, a bio-information estimation model, a bio-information estimation result, and the like, to and from the external device. In this case, the external device may include an information processing device such as a smartphone, a tablet personal computer (PC), a desktop computer, a laptop computer, and the like. In this case, the communication interface 630 may communicate with the external device by using various wired or wireless communication techniques, such as Bluetooth communication, Bluetooth Low Energy (BLE) communication, Near Field Communication (NFC), wireless local area network (WLAN) communication, Zigbee communication, Infrared Data Association (IrDA) communication, wireless fidelity (Wi-Fi) Direct (WFD) communication, Ultra-Wideband (UWB) communication, Ant+ communication, Wi-Fi communication, Radio Frequency Identification (RFID) communication, 3G communication, 4G communication, 5G communication, and the like. However, this is merely exemplary and is not intended to be limiting.

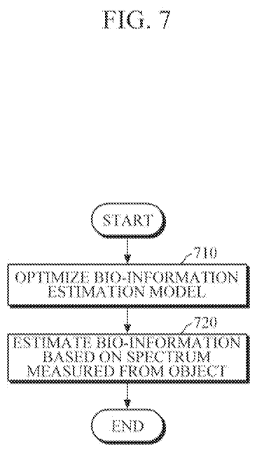

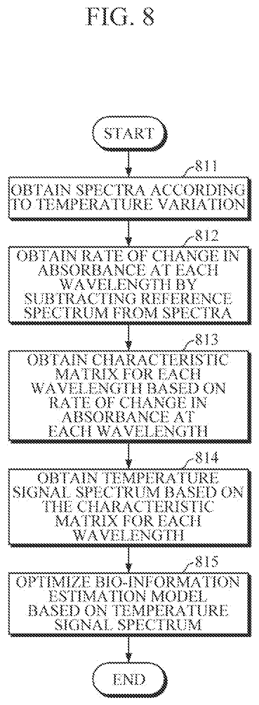

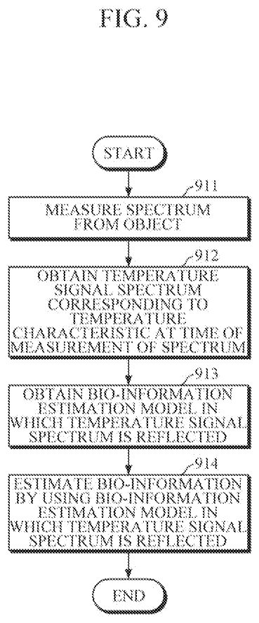

FIG. 7 is a flowchart illustrating a method of estimating bio-information according to an embodiment of the present disclosure. FIG. 8 is a diagram illustrating an example of optimizing a bio-information estimation model. FIGS. 9 to 11 are diagrams illustrating examples of estimating bio-information. The embodiments of FIGS. 7 to 11 may be performed by the aforementioned apparatuses 100 and 600 for estimating bio-information, which are described above in detail, and thus will be briefly described below in order to avoid redundancy.

The apparatuses 100 and 600 for estimating bio-information may optimize a bio-information estimation model by performing calibration in operation 710.

An example of optimizing a bio-information estimation model will be described below with reference to FIG. 8.

The apparatuses 100 and 600 for estimating bio-information may obtain a plurality of first spectra in a changing temperature environment in operation 811.

The apparatuses 100 and 600 for estimating bio-information may obtain rates of change in absorbance at each wavelength according to a temperature variation by subtracting a reference spectrum from the plurality of first spectra in operation 812. In this case, the reference spectrum may be a spectrum measured when a user is in a fasting state, a spectrum measured in an aqueous solution, any one spectrum, such as a first spectrum measured at a specific time among the plurality of spectra obtained in operation 811, an average of all the first spectra, and the like.

The apparatuses 100 and 600 for estimating bio-information may obtain characteristic data for each wavelength based on the rates of change in absorbance at each wavelength according to the temperature variation in operation 813. For example, the apparatuses 100 and 600 for estimating bio-information may convert the variations in absorbance at each wavelength according to the temperature variation into vectors, and may generate characteristic matrices for each wavelength according to the temperature variation by using the absorbance change vectors at each wavelength.

The apparatuses 100 and 600 for estimating bio-information may obtain first temperature signal spectra according to the temperature variation, based on the characteristic matrices for each wavelength in operation 814. For example, the apparatuses 100 and 600 for estimating bio-information may select a row/column, at which a degree of change in spectrum is greatest, from the characteristic matrices for each wavelength according to each temperature variation characteristic, and may obtain values in the selected row/column as the first temperature signal spectra.

The apparatuses 100 and 600 for estimating bio-information may optimize a bio-information estimation model based on the first temperature signal spectra in operation 815. For example, by reflecting the first temperature signal spectrum in a reference bio-information estimation model with no temperature variation characteristics being reflected therein, the apparatuses 100 and 600 for estimating bio-information may obtain an optimized bio-information estimation model.

Referring to FIG. 7, the apparatuses 100 and 600 for estimating bio-information may estimate bio-information in operation 720 by using the characteristic data for each wavelength, generated in operation 710, a pure temperature signal spectrum, and/or the optimized bio-information estimation model.

Various examples of the estimating of bio-information in operation 720 will be described below with reference to FIGS. 9 to 11.

Referring to FIG. 9, in response to a request for estimating bio-information, the apparatuses 100 and 600 for estimating bio-information may control the spectrum measurer to measure a second spectrum from an object in operation 911.

The apparatuses 100 and 600 for estimating bio-information may obtain a second temperature signal spectrum corresponding to a temperature characteristic at the time of measurement of the second spectrum in operation 912. For example, among the plurality of first temperature signal spectra measured according to a temperature variation, the apparatuses 100 and 600 for estimating bio-information may obtain the second temperature signal spectrum by using a similarity or variance between the plurality of first temperature signal spectra and the second spectrum, or by using statistical test methods, and the like.

The apparatuses 100 and 600 for estimating bio-information may obtain a bio-information estimation model in operation 913, in which the obtained second temperature signal spectrum is reflected, and may estimate bio-information by using the obtained bio-information estimation model in operation 914. For example, the apparatuses 100 and 600 for estimating bio-information may extract a bio-information estimation model, in which the second temperature signal spectrum is reflected, from the temperature variation characteristic DB 230, and may estimate bio-information based on the second spectrum by using the extracted bio-information estimation model.

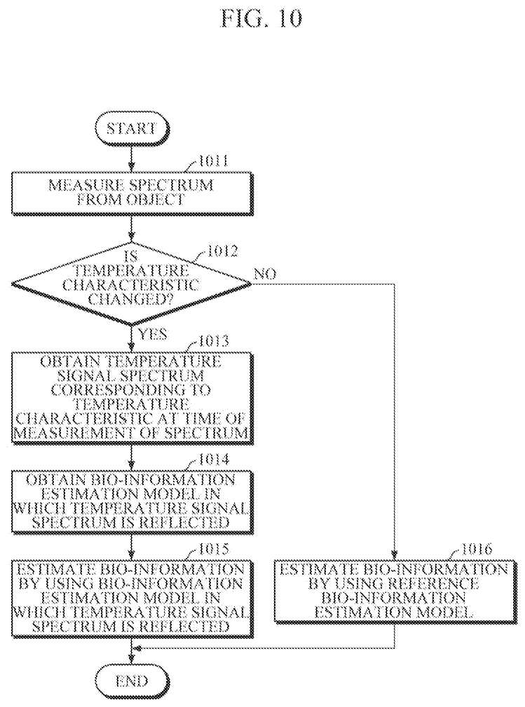

Referring to FIG. 10, the apparatuses 100 and 600 for estimating bio-information may measure the second spectrum from the object in operation 1011, and may detect whether a temperature characteristic at the time of measurement of the second spectrum is changed compared to the reference time in operation 1012. For example, the apparatuses 100 and 600 for estimating bio-information may generate characteristic matrices for each wavelength, representative of the temperature characteristic at the time of measurement of the second spectrum, by using the second spectrum and the reference spectrum, and may detect whether the temperature characteristic is changed by analyzing the generated characteristic matrices for each wavelength.

Based on detecting the change in temperature characteristic in operation 1012, the apparatuses 100 and 600 for estimating bio-information may obtain the second temperature signal spectrum corresponding to the temperature characteristic at the time of measurement of the second spectrum in operation 1013: may obtain a bio-information estimation model in which the second temperature signal spectrum is reflected in operation 1014; and may estimate bio-information by using the obtained bio-information estimation model operation 1015. Based on detecting no change in temperature characteristic in operation 1012, the apparatuses 100 and 600 for estimating bio-information may estimate bio-information by using a reference bio-information estimation model in operation 1016.

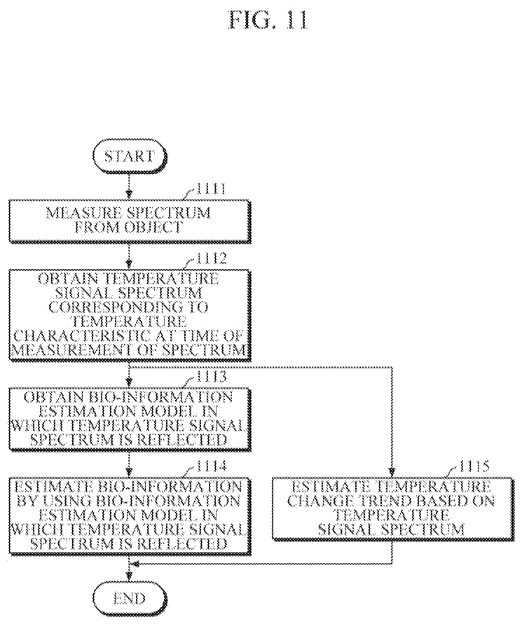

Referring to FIG. 11, in the same manner as the embodiment of FIG. 9, the apparatuses 100 and 600 for estimating bio-information may measure a second spectrum from an object in operation 1111, may obtain a second temperature signal spectrum, corresponding to a temperature characteristic at the time of measurement of the second spectrum, in operation 1112, may obtain a bio-information estimation model in operation 1113, in which the obtained second temperature signal spectrum is reflected, and may estimate bio-information by using the obtained bio-information estimation model in operation 1114. Further, the apparatuses 100 and 600 for estimating bio-information may estimate a relative temperature change trend at the time of bio-information estimation compared to a reference time in operation 1115.

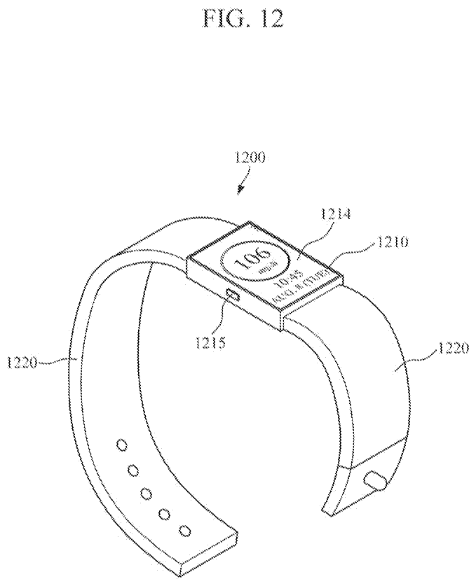

FIG. 12 is a wearable device according to an embodiment of the present disclosure. Various embodiments of the apparatuses 100 and 600 for estimating bio-information may be mounted in a wearable device such as a smart band or a smart watch as illustrated in FIG. 12, but the apparatuses 100 and 600 for estimating bio-information is not limited thereto and may be mounted in a smart device, such as a smartphone, a tablet PC, smart earphones, smart glasses, and the like, or in an information processing device such as a desktop computer, a laptop computer, and the like.

Referring to FIG. 12, the wearable device 1200 includes a main body 1210 and a strap 1220.

The main body 1210 may be worn on a user's wrist with the strap 1220. The main body 1210 may include various modules to perform various functions of the wearable device 1200. A battery may be embedded in the main body 1210 or the strap 1220 to supply power to the various modules of the wearable device 1200. The strap 1220 may be connected to both ends of the main body 1210, and may be flexible so as to be wrapped around a user's wrist. The strap 1220 may be composed of a first strap and a second strap which are separated from each other. Respective ends of the first strap and the second strap are connected to the main body 1210, and the other ends thereof may be connected to each other via a connecting means. In this case, the connecting means may be formed as magnetic connection, Velcro connection, pin connection, and the like, but is not limited thereto. Further, the strap 1220 is not limited thereto, and may be integrally formed as a non-detachable band.

The main body 1210 may include a spectrum measurer. As described above, the spectrum measurer includes a light source and a detector, and may measure spectra from a user.

A processor may be mounted in the main body 1210. The processor may be electrically connected to various modules of the wearable device 1200. The processor may obtain characteristic data for each wavelength according to a temperature variation by using a plurality of spectra measured in various changing temperature environments, and may optimize a bio-information estimation model by using the obtained characteristic data for each wavelength. In addition, the processor may estimate bio-information by using spectra measured from a user, the characteristic data for each wavelength, and/or the optimized bio-information estimation model.

Further, the main body 1210 may include a storage which stores a variety of reference information and information processed by the various modules.

In addition, the main body 1210 may include a manipulator 1215 which is provided on one side surface of the main body 1210, and receives a user's control command and transmits the received control command to the processor. The manipulator 1215 may have a power button to input a command to turn on/off the wearable device 1200.

Further, a display 1214 for outputting information to a user may be mounted on a front surface of the main body 1210. The display 1214 may have a touch screen for receiving touch input. The display may receive a user's touch input and transmit the touch input to the processor, and may display processing results of the processor.

Moreover, the main body 1210 may include a communication interface for communication with an external device. The communication interface may transmit a blood glucose estimation result to the external device, such as a user's smartphone, and may obtain a unit spectrum of a blood glucose signal from an apparatus for obtaining a blood glucose signal spectrum.

Example embodiments of the present disclosure may be implemented by computer-readable code written on a non-transitory computer-readable medium and executed by a processor. The computer-readable medium may be any type of recording device in which data is stored in a computer-readable manner.

Examples of the computer-readable medium include a ROM, a RAM, a CD-ROM, a magnetic tape, a floppy disc, an optical data storage, and a carrier wave (e.g., data transmission through the Internet). The computer-readable medium can be distributed over a plurality of computer systems connected to a network so that computer-readable code is written thereto and executed therefrom in a decentralized manner. Functional programs, code, and code segments needed for implementing example embodiments of the present disclosure can be readily deduced by programmers of ordinary skill in the art to which the present disclosure pertains.

The present disclosure has been described herein with regard to example embodiments. However, it will be obvious to those skilled in the art that various changes and modifications can be made without changing the technical concepts of the present disclosure. Thus, it is clear that the above-described embodiments are illustrative in all aspects and are not intended to limit the present disclosure.

What is claimed is:

1. A method of optimizing a bio-information estimation model, the method comprising:
obtaining a plurality of spectra according to a temperature variation;
obtaining a rate of change in absorbance at each wavelength of the plurality of spectra according to the temperature variation by subtracting a reference spectrum from the plurality of spectra;
generating a characteristic matrix for each wavelength based on the rate of change in absorbance at each wavelength according to the temperature variation;
obtaining a temperature signal spectrum based on the characteristic matrix for each wavelength; and
optimizing the bio-information estimation model based on the temperature signal spectrum.

2. The method of claim 1, further comprising removing noise from the plurality of spectra based on at least one of differentiation, filtering, asymmetric least square (ALS), detrend, multiplicative scatter correction (MSC), extended multiplicative scatter correction (EMSC), standard normal variate (SNV), mean centering (MC), Fourier transform (FT), orthogonal signal correction (OSC), and Savitzky-Golay (SG) smoothing.

3. The method of claim 1, wherein the reference spectrum comprises at least one among any one of the plurality of spectra, an average of the plurality of spectra, a spectrum measured in a fasting state, and a spectrum measured in an aqueous solution.

4. The method of claim 1, wherein the generating of the characteristic matrix for each wavelength comprises converting the rate of change in absorbance at each wavelength according to the temperature variation into vectors, and generating the characteristic matrix for each wavelength according to the temperature variation by sequentially calculating an inner product between an absorbance change vector at a specific wavelength and absorbance change vectors at all wavelengths.

5. The method of claim 1, wherein the obtaining of the temperature signal spectrum comprises selecting a row or a column from the characteristic matrix for each wavelength according to the temperature variation, and obtaining a spectrum of the row or the column as the temperature signal spectrum.

6. The method of claim 5, wherein the obtaining of the temperature signal spectrum comprises selecting the row or the column from the characteristic matrix for each wavelength according to the temperature variation, based on a degree of change in spectrum of each row and column of the characteristic matrix.

7. The method of claim 6, wherein the obtaining of the temperature signal spectrum comprises selecting the row or the column, at which a degree of change in spectrum is greatest, from the characteristic matrix for each wavelength according to the temperature variation.

8. The method of claim 1, wherein the optimizing of the bio-information estimation model comprises obtaining a plurality of bio-information estimation models optimized according the temperature variation, by updating the bio-information estimation model based on the temperature signal spectrum according to the temperature variation.

9. The method of claim 1, further comprising generating the bio-information estimation model based on the plurality of spectra.

10. The method of claim 1, wherein the bio-information estimation model is based on classical least square (CLS) or net analyte signal (NAS).

11. An apparatus for estimating bio-information, the apparatus comprising:
a spectrum measurer configured to measure a spectrum from an object of a user; and
a processor configured to:
obtain a temperature signal spectrum, corresponding to a temperature characteristic at a time of measurement of the spectrum, based on characteristic data for each wavelength of the spectrum according to a temperature variation; and
estimate the bio-information based on the spectrum by using a bio-information estimation model in which the temperature signal spectrum is reflected.

12. The apparatus of claim 11, wherein the spectrum measurer comprises:
a light source configured to emit light onto the object; and
a detector configured to detect light reflected by or scattered from the object.

13. The apparatus of claim 11, wherein the processor is further configured to obtain the temperature signal spectrum, corresponding to the temperature characteristic at the time of measurement of the spectrum, based on at least one of a similarity and a variance between a plurality of temperature signal spectra, obtained based on the characteristic data for each wavelength, and the measured spectrum, or by using a result of a statistical test.

14. The apparatus of claim 13, wherein the similarity comprises at least one of Euclidean distance, Pearson correlation coefficient, Spearman correlation coefficient, and Cosine similarity.

15. The apparatus of claim 13, wherein the statistical test comprises at least one of t-test, z-test, and ANOVA test.

16. The apparatus of claim 11, wherein the processor is further configured to detect a change in the temperature characteristic at the time of measurement of the spectrum, and based on detecting the change in the temperature characteristic, obtain the temperature signal spectrum corresponding to the temperature characteristic at the time of measurement of the spectrum.

17. The apparatus of claim 16, wherein the processor is further configured to detect the change in the temperature characteristic, based on at least one of whether there is a non-temperature dependent wavelength in a specific wavelength range, whether a non-temperature dependent wavelength is shifted according to an increase in the temperature variation, and whether a range of wavelengths shorter than a first wavelength increases and a range of wavelengths longer than a second wavelength decreases.

18. The apparatus of claim 11, wherein the processor is further configured to obtain the bio-information estimation model, in which the temperature signal spectrum is reflected, by updating a reference bio-information estimation model based on the temperature signal spectrum corresponding to the temperature characteristic at the time of measurement of the spectrum.

19. The apparatus of claim 11, wherein the processor is further configured to obtain a plurality of spectra as training data from the user, and generate the characteristic data for each wavelength based on the training data.

20. The apparatus of claim 19, wherein the processor is further configured to obtain a rate of change in absorbance at each wavelength by subtracting a reference spectrum from each of the plurality of spectra, and generate the characteristic data for each wavelength based on the obtained rate of change in absorbance at each wavelength.

21. The apparatus of claim 11, wherein the processor is further configured to estimate a relative temperature change trend at the time of measurement of the spectrum compared to a reference time, based on the temperature signal spectrum corresponding to the temperature characteristic at the time of measurement of the spectrum.

22. The apparatus of claim 11, wherein the bio-information comprises one or more of an antioxidant-related substance, blood glucose, triglyceride, cholesterol, calories, protein, carotenoid, lactate, and uric acid.

23. A method of estimating bio-information, the method comprising:
measuring a spectrum from an object of a user;
obtaining a temperature signal spectrum, corresponding to a temperature characteristic at a time of measurement of the spectrum, based on characteristic data for each wavelength of the spectrum according to a temperature variation; and
estimating the bio-information based on the spectrum by using a bio-information estimation model in which the obtained temperature signal spectrum is reflected.

24. The method of claim 23, wherein the obtaining of the temperature signal spectrum comprises obtaining the temperature signal spectrum, corresponding to the temperature characteristic at the time of measurement of the spectrum, based on at least one of a similarity and a variance between a plurality of temperature signal spectra, obtained based on the characteristic data for each wavelength according to the temperature variation, and the spectrum, or by using a result of a statistical test.

25. The method of claim 23, wherein the obtaining of the temperature signal spectrum comprises detecting a change in the temperature characteristic at the time of measurement of the spectrum, and based on detecting the change in the temperature characteristic, obtaining the temperature signal spectrum.

26. The method of claim 25, wherein the detecting of the change in the temperature characteristic comprises detecting the change in the temperature characteristic, based on at least one of whether there is a non-temperature dependent wavelength in a specific wavelength range, whether a non-temperature dependent wavelength is shifted according to an increase in temperature variation, and whether a range of wavelengths shorter than a first wavelength increases and a range of wavelengths longer than a second wavelength decreases.

27. The method of claim 23, further comprising obtaining the bio-information estimation model, in which the temperature signal spectrum is reflected, by updating a reference bio-information estimation model based on the temperature signal spectrum corresponding to the temperature characteristic at the time of measurement of the spectrum.

28. The method of claim 23, further comprising:
obtaining a plurality of spectra, measured from the user, as training data; and
generating the characteristic data for each wavelength based on the training data.

29. The method of claim 28, wherein the generating of the characteristic data for each wavelength comprises:
obtaining a rate of change in absorbance at each wavelength by subtracting a reference spectrum from each of the plurality of spectra; and
generating the characteristic data for each wavelength based on the obtained rate of change in absorbance at each wavelength.

30. The method of claim 23, further comprising estimating a relative temperature change trend at the time of measurement of the spectrum compared to a reference temperature, based on the temperature signal spectrum corresponding to the temperature characteristic at the time of measurement of the spectrum.

31. A method estimating bio-information, the method comprising:
obtaining a spectrum from an object of a user;
obtaining a temperature signal spectrum, corresponding to a temperature characteristic at a time of measurement of the spectrum, based on characteristic data for each wavelength of the spectrum according to a temperature variation;
updating a bio-information estimation model based on the temperature signal spectrum corresponding to the temperature characteristic at the time of measurement of the spectrum; and
estimating the bio-information based on the spectrum by using the bio-information estimation model, based on updating the bio-information estimation model.
